(19) United States
(12) Patent Application Publication
(10) Pub. No.: US 2023/0045199 A1
(43) Pub. Date: Feb. 9, 2023

(54) COMPOSITIONS AND METHODS FOR HEMOGLOBIN PRODUCTION

(71) Applicants: THE CHILDREN'S HOSPITAL OF PHILADELPHIA, Philadelphia, PA (US); THE TRUSTEES OF THE UNIVERSITY OF PENNSYLVANIA, Philadelphia, PA (US)

(72) Inventors: Gerd BLOBEL, Bala Cynwyd, PA (US); Scott PESLAK, King of Prussia, PA (US); Junwei SHI, Philadelphia, PA (US)

(21) Appl. No.: 17/786,147

(22) PCT Filed: Dec. 23, 2020
(86) PCT No.: PCT/US20/66779

§ 371 Date: Jun. 16, 2022

Related U.S. Application Data
(60) Provisional application No. 62/952,653, filed on Dec. 23, 2019.

(51) Int. Cl. C12N 15/113; A61P 7/06; A61K 31/454; A61K 31/713

(52) U.S. Cl. CPC C12N 15/1137; A61P 7/06; A61K 31/454; A61K 31/713; C12N 2310/20; C12N 2320/31

(57) ABSTRACT

Methods and compositions for producing fetal hemoglobin and treating a hemoglobinopathy or thalassemia are disclosed.

This application claims priority under 35 U.S.C. § 119(e) to U.S. Provisional Patent Application No. 62/952,653, filed Dec. 23, 2019. The foregoing application is incorporated by reference herein.

This invention was made with government support under Grant Nos. R01DK054937, R01HL119479, and T32HL007439 awarded by National Institutes of Health. The government has certain rights in the invention.

### FIELD OF THE INVENTION

The present invention relates to the field of hematology. More specifically, the invention provides compositions and methods for the production of various forms of hemoglobin, including adult and fetal type hemoglobin.

### BACKGROUND OF THE INVENTION

Several publications and patent documents are cited throughout the specification in order to describe the state of the art to which this invention pertains. Each of these citations is incorporated herein by reference as though set forth in full.

Sickle cell disease and thalassemia cause significant worldwide morbidity and mortality (Modell et al. (2008) Bull. World Health Org., 86:480-487; Modell et al. (2008) J. Cardiovasc. Magn. Reson., 10:42). However, effective drugs do not exist for these illnesses. One goal in the treatment of these diseases is to reactivate fetal hemoglobin (HbF). HbF reduces the propensity of sickle cell disease red blood cells to undergo sickling. Indeed, high fetal globin levels are associated with improved outcomes for sickle cell anemia patients (Platt et al. (1994) N. Engl. J. Med., 330: 1639-1644). Elevating HbF also reduces the globin chain imbalance in certain thalassemias, thereby improving symptoms. There is an enormous unmet need to identify compounds that ameliorate the course of these diseases.

### SUMMARY OF THE INVENTION

In accordance with the present invention, compositions and methods are provided for increasing hemoglobin levels (e.g., fetal hemoglobin) in a cell or subject. In a particular embodiment, the method comprises administering at least one protein phosphatase 6 catalytic subunit (PP6C) inhibitor to the cell or subject. In a particular embodiment, the subject has a hemoglobinopathy or thalassemia. In a particular embodiment, the cell is an erythroid cell. In a particular embodiment, the PP6C inhibitor is a small molecule. The PP6C inhibitor may be, for example, a phosphatase inhibitor or a heme binding domain inhibitor. The method may further comprise delivering at least one fetal hemoglobin inducer to the cell or subject.

In accordance with another aspect of the instant invention, methods of inhibiting, treating, and/or preventing a hemoglobinopathy (e.g., sickle cell disease) or thalassemia in a subject are provided. In a particular embodiment, the method comprises administering at least one PP6C inhibitor to a subject in need thereof. The PP6C inhibitor may be in a composition with a pharmaceutically acceptable carrier. In a particular embodiment, the subject has a β-chain hemoglobinopathy. In a particular embodiment, the subject has sickle cell anemia. In a particular embodiment, the PP6C inhibitor is a small molecule. The PP6C inhibitor may be, for example, a phosphatase inhibitor. The method may further comprise delivering at least one other fetal hemoglobin inducer to the subject.

### BRIEF DESCRIPTIONS OF THE DRAWINGS

FIG. 1A outlines the identification of PP6C as a novel regulator of fetal hemoglobin utilizing a domain-focused phosphatase CRISPR screening approach. The phosphatase domain sgRNA library was cloned into an sgRNA2.1 scaffold and introduced into human umbilical cord blood-derived erythroid progenitor cells (HUDEP-2) expressing Cas9. Anti-HbF fluorescence-activated cell sorting (FACS) was used to isolate the top 10% and bottom 10% of HbF expressing cells and sgRNAs were deep sequenced. FIG. 1B illustrates the results of this CRISPR genetic screen with sgRNA representation in low-HbF versus high-HbF populations as log 2-transformed normalized read counts. Depletion of PP6C results in significant HbF enrichment in 5 of the 6 sgRNA tested as compared to non-targeting control sgRNA.

FIGS. 2A-2D provide data regarding validation of PP6C as a regulator of HbF in human primary cells utilizing RNP-based electroporation of SpCas9 with guide RNA targeting PP6C. FIG. 2A depicts protein levels by Western blot of PP6C and gamma-globin with 6 independent guide RNA targeting PP6C or a non-targeting guide RNA. Protein levels of PP6C were depleted more than 85% utilizing PP6C sgRNA #1 and #3, which correlated with significantly increased gamma-globin levels. These specific guide RNAs were utilized for further primary human cell testing. FIG. 2B provides a graph of gamma globin mRNA levels in four independent biological replicates of human primary cells treated with PP6C sgRNA #1 or #3; a guide RNA targeting the BCL11A +58 enhancer (positive control), or a non-targeting guide RNA (negative control). FIG. 2C provides representative flow cytometric plots of HbF staining (top) and compiled F-cell flow cytometric staining in four independent biological replicates (bottom) in cells treated with PP6C sgRNA, BCL11A +58 sgRNA, or non-targeting sgRNA. The percentage displayed is the proportion of HbF-positive cells (F-cells). FIG. 2D are representative cation-exchange high performance liquid chromatography (HPLC) tracings (top) and complied HPLC HbF+ quantitation in two independent biological replicates (bottom) in cells treated with PP6C sgRNA, BCL11A +58 sgRNA, or non-targeting sgRNA. HbF elutes as a distinct peak (grey box), followed by adult hemoglobin (HbA). Percentages are quantified as HbF percentage of total globins (HbF+HbA).

FIG. 3A provides the canonical amino acid sequence (SEQ ID NO: 1) of PP6C (UniProt Identifier O00743-1) (isoform b). FIG. 3B provides a nucleotide sequence (SEQ ID NO: 2) which encodes SEQ ID NO: 1. FIG. 3C provides the nucleotide sequence (SEQ ID NO: 3) of the PP6C transcript variant 1 mRNA (NCBI Reference Sequence: NM_001123355.1), which is the longest transcript of PP6C that encodes the longest isoform (isoform a). All splice variants and all forms, native or processed are embodied in this invention.

### DETAILED DESCRIPTION OF THE INVENTION

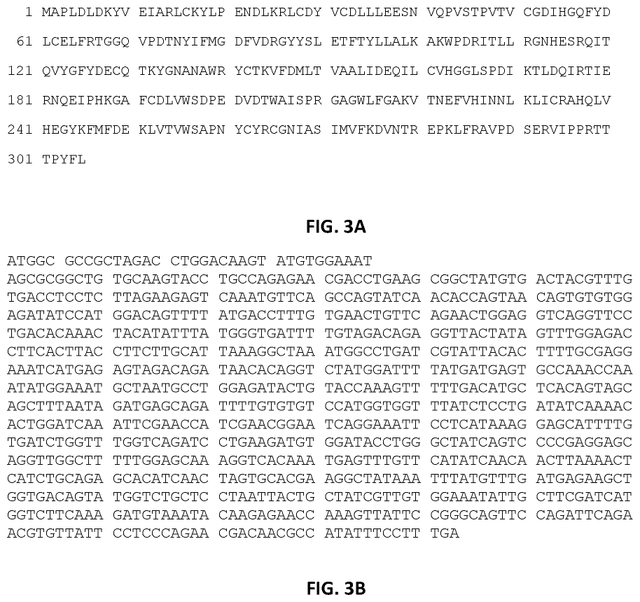

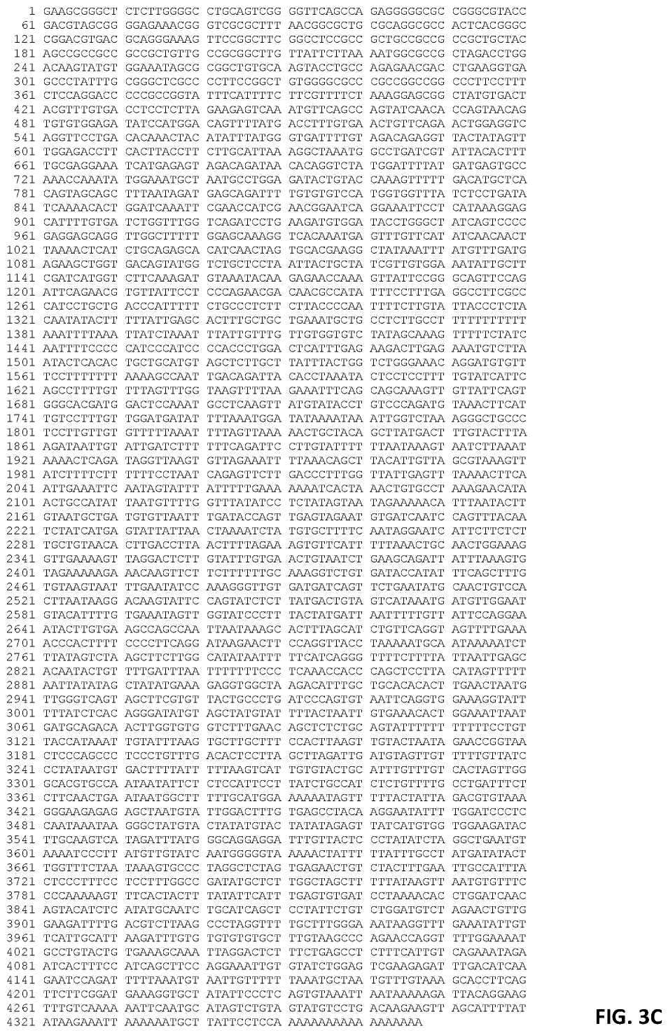

A major goal in the treatment of sickle cell disease and thalassemia is the reactivation of fetal type globin expression in cells of the adult red blood lineage. In an unbiased genetic screen, protein phosphatase 6 catalytic subunit (PP6C, also known as PPP6C) was identified as a strong regulator of fetal globin production. PP6C (see, e.g., PubMed GeneID: 5537; UniProt Identifier 000743; GenBank Accession Nos. NM_001123355.1, NP_001116827.1 (isoform a), NM_001123369.1, NP_001116841.1 (isoform c), NM_002721.5, and NP_002712.1 (isoform b)) is a catalytic subunit of protein phosphatase 6 (PP6), a serine/threonine protein phosphatase that de-phosphorylates the protein kinase GCN2 among other proteins and, thus, indirectly regulates protein translation (Bastians et al. (1996) J. Cell Sci. 109(12):2865-74; Cherkasova et al. Genes. Dev (2003) 17(7):859-72; Stefansson et al. J. Biol Chem (2006) 281(32):22624-34; Morales-Johansson et al. PLoS One (2009) 4(7):e6331; Hosing et al. J Biol Chem (2012) 287(12):9230-9; Wengrod et al. Sci Signal (2015) 8(367):ra27). All splice variants and all forms or isoforms, native or processed, are encompassed by the instant invention. For example, isoforms a, b, and c of PP6C are encompassed by the instant invention. FIG. 3A provides an amino acid sequence (SEQ ID NO: 1) of PP6C (UniProt Identifier 000743-1). FIG. 3B provides a nucleotide sequence (SEQ ID NO: 2) which encodes SEQ ID NO: 1. FIG. 3C provides the nucleotide sequence (SEQ ID NO: 3) of the PP6C transcript variant 1 mRNA (NCBI Reference Sequence: NM_001123355.1), which is the longest transcript of PP6C that encodes the longest isoform.

Herein, it is shown that the loss of PP6C increases fetal hemoglobin production in human erythroid cells, including primary cells. Without being bound by theory, the mechanism by which this occurs likely involves transcriptional upregulation of fetal hemoglobin production. This role is exploited herein to treat hemoglobinopathies such as sickle cell anemia and thalassemia. For example, as a protein phosphatase, PP6C has a catalytic domain that can be inhibited (e.g., by a small molecule). In addition, PP6C can be removed by targeted degradation or inhibition.

In accordance with the instant invention, compositions and methods are provided for increasing hemoglobin production in a cell or subject. In a particular embodiment, the method increases fetal hemoglobin and/or embryonic globin expression, particularly fetal hemoglobin. The method comprises administering at least one PP6C inhibitor to the cell, particularly an erythroid precursor cell or erythroid cell, or subject. In a particular embodiment, the subject has a hemoglobinopathy (such as sickle cell disease) or thalassemia. In a particular embodiment, the subject has sickle cell anemia. In a particular embodiment, the subject has a thalassemia, particularly a β-thalassemia, and more particularly major β-thalassemia.

The PP6C inhibitor may be administered in a composition further comprising at least one pharmaceutically acceptable carrier. In a particular embodiment, the method further comprises any means by which to induce fetal hemoglobin, such as administering at least one other fetal hemoglobin inducer. Fetal hemoglobin inducers include, without limitation, a lysine-specific demethylase 1 (LSD1) inhibitor (e.g., RN-1 and tranylcypromine (TCP) (Cui et al. (2015) Blood 126(3):386-96; Shi et al. (2013) Nat. Med., 19(3): 291-294; Sun et al. (2016) Reprod. Biol. Endocrinol., 14:17)), pomalidomide (Moutouh-de Parseval et al. (2008) J. Clin. Invest., 118(1):248-258; Dulmovits et al., Blood (2016) 127(11):1481-92), hydroxyurea (Charache et al., NEJM (1995) 332(20):1317-22), 5-azacytidine (Humphries et al., J. Clin. Invest. (1985) 75(2):547-57), sodium butyrate, activators (inducer) of the FOXO3 pathway (e.g., metformin, phenformin, or resveratrol; Zhang et al., Blood (2018) 132(3): 321-333), histone methyltransferase (HMT) inhibitors (e.g., a histone lysine methyltransferase inhibitor, euchromatic histone-lysine N-methyltransferase 2 (EHMT2; G9a) inhibitor, euchromatic histone-lysine N-methyltransferase 1 (EHMT1; G9a-like protein (GLP)) inhibitor, UNC0638 (2-cyclohexyl-N-(1-isopropylpiperidin-4-yl)-6-methoxy-7-(3-(pyrrolidin-1-yl)propoxy) quinazolin-4-amine) (Renneville et al., Blood (2015) 126(16):1930-9; Krivega et al., Blood (2015) 126(5):665-72), chaetocin, BIX-01294, UNC 0224, UNC 0642, UNC 0631, UNC 0646, A-366 (Sweis et al. (2014) ACS Med. Chem. Lett., 5(2):205-209), etc.), histone deacetylase (HDAC) inhibitors (e.g., entinostat; Bradner et al., PNAS (2010) 107(28):12617-22), and eIF2αK1 inhibitors (see, e.g., PCT/US18/15918). In a particular embodiment, the fetal hemoglobin inducer is pomalidomide, UNC0638, or hydroxyurea, particularly pomalidomide, or similar imide. In a particular embodiment, the fetal hemoglobin inducer is pomalidomide or hydroxyurea, particularly pomalidomide or similar imide. The PP6C inhibitor and the fetal hemoglobin inducer can be delivered to the cell or subject sequentially or consecutively (e.g., in different compositions) and/or at the same time (e.g., in the same composition).

In accordance with another aspect of the instant invention, compositions and methods for inhibiting (e.g., reducing or slowing), treating, and/or preventing a hemoglobinopathy (e.g., γ-hemoglobinopathy) or thalassemia in a subject are provided. In a particular embodiment, the methods comprise administering to a subject in need thereof a therapeutically effective amount of at least one PP6C inhibitor. The PP6C inhibitor may be administered in a composition further comprising at least one pharmaceutically acceptable carrier. In a particular embodiment, the subject has β-thalassemia or sickle cell anemia. In a particular embodiment, the subject has sickle cell anemia.

The methods of the instant invention may comprise administering at least two different PP6C inhibitors (e.g., two PP6C inhibitors with two different mechanisms of action are administered), particularly at least one phosphatase domain inhibitor and at least one inhibitor that targets another domain of PP6C and/or PP6. In a particular embodiment, the method further comprises administering at least one other fetal hemoglobin inducer to the subject as described hereinabove. In a particular embodiment, the fetal hemoglobin inducer is pomalidomide or hydroxyurea, particularly pomalidomide. The PP6C inhibitor and the fetal hemoglobin inducer can be administered to the subject sequentially or consecutively (e.g., in different compositions) and/or at the same time (e.g., in the same composition).

PP6C inhibitors are compounds which reduce PP6 and/or PP6C activity (e.g., phosphatase activity), inhibit or reduce PP6 and/or PP6C-substrate/partner interaction, and/or the expression of PP6 and/or PP6C. In a particular embodiment, the PP6C inhibitor inhibits or degrades a co-factor, or regulatory subunit. In a particular embodiment, the PP6C inhibitor is specific and/or targeted to PP6C. In a particular embodiment, the PP6C inhibitor reduces PP6C activity and/or expression to greater levels than other eukaryotic phosphatases. Examples of PP6C inhibitors include, without limitation, proteins, polypeptides, peptides, antibodies, small molecules, and nucleic acid molecules. In a particular embodiment, the PP6C inhibitor is a phosphatase inhibitor (e.g., a Ser/Thr protein phosphatase inhibitor (e.g., a type 2A protein phosphatase family inhibitor) or phosphoprotein phosphatase (PPP) inhibitor). In a particular embodiment, the PP6C inhibitor is an inhibitory nucleic acid molecule, such as an antisense, microRNA, siRNA, or shRNA molecule (or a nucleic acid molecule encoding the inhibitory nucleic acid molecule). For example, microRNA-31 (miR-31) reduces expression of PP6C (Yan et al., Nature Communications (2015) 6:7652). In a particular embodiment, the inhibitory nucleic acid molecule targets a sequence within the phosphatase domain. In a particular embodiment, the inhibitory nucleic acid molecule targets a sequence or comprises a sequence (inclusive of RNA version of DNA molecules) as set forth in the Example (e.g., Table 1) provided herein (e.g., sgRNA1, sgRNA2, sgRNA3, sgRNA4, sgRNA5, or sgRNA6; particularly, sgRNA1, sgRNA3, or sgRNA4; particularly, sgRNA1 or sgRNA3). In a particular embodiment, the inhibitory nucleic acid molecule targets a sequence or comprises a sequence (e.g., RNA version) which has at least 80%, 85%, 90%, 95%, 97%, 99%, or 100% homology or identity to a sequence set forth in the Example (e.g., sgRNA1, sgRNA2, sgRNA3, sgRNA4, sgRNA5, or sgRNA6; particularly, sgRNA1, sgRNA3, or sgRNA4; particularly, sgRNA1 or sgRNA3). The sequences may be extended or shortened by 1, 2, 3, 4, or 5 nucleotides at the end of the sequence (e.g., the extended sequence may correspond to the genomic sequence). In a particular embodiment, the PP6C inhibitor is a CRISPR based targeting of the PP6C gene (e.g., with a guide RNA targeting the PP6C gene). In a particular embodiment, the PP6C inhibitor is a small molecule. The PP6C inhibitor may be a synthetic or non-natural compound.

A variety of inhibitors to the phosphoprotein phosphatase (PPP) family of serine/threonine protein phosphatases are known in the art, including, without limitation: okadaic acid (Bialojan et al., Biochem J. (1988) 256(1):283-90), fostriecin and microcystin-LR (Hastie et al., FEBS Letters (1998) 431(3):357-61), calyculin A (Ishihara et al., Biochem Biophys Res Commun (1989) 159(3):871-7), cantharidin (Li et al., PNAS (1992) 89(24):11867-70; Honkanen et al. FEBS Letters (1993) 330(3):283-6), and nodularin and tautomycin (Swingle et al., Methods Mol Biol. (2007) 365:23-38.). More recently, these compounds were shown to also inhibit PP6 (Prickett et al., J. Biol Chem (2006) 281(41):30503-11). Thus, examples of PP6 or PP6C inhibitors include, without limitation, okadaic acid, fostriecin, microcystin-LR, calyculin A, cantharidin, nodularin and tautomycin.

Clustered, regularly interspaced, short palindromic repeat (CRISPR)/Cas9 (e.g., from Streptococcus pyogenes) technology and gene editing are well known in the art (see, e.g., Sander et al. (2014) Nature Biotech., 32:347-355; Jinek et al. (2012) Science, 337:816-821; Cong et al. (2013) Science 339:819-823; Ran et al. (2013) Nature Protocols 8:2281-2308; Mali et al. (2013) Science 339:823-826; addgene.org/crispr/guide/). The RNA-guided CRISPR/Cas9 system involves expressing Cas9 along with a guide RNA molecule (gRNA). When coexpressed, gRNAs bind and recruit Cas9 to a specific genomic target sequence where it mediates a double strand DNA (dsDNA) break. The binding specificity of the CRISPR/Cas9 complex depends on two different elements. First, the binding complementarity between the targeted genomic DNA (genDNA) sequence and the complementary recognition sequence of the gRNA (e.g., ˜18-22 nucleotides, particularly about 20 nucleotides). Second, the presence of a protospacer-adjacent motif (PAM) juxtaposed to the genDNA/gRNA complementary region (Jinek et al. (2012) Science 337:816-821; Hsu et al. (2013) Nat. Biotech., 31:827-832; Sternberg et al. (2014) Nature 507:62-67). The PAM motif for S. Pyogenes Cas9 has been fully characterized, and is NGG or NAG (Jinek et al. (2012) Science 337:816-821; Hsu et al. (2013) Nat. Biotech., 31:827-832). Other PAMs of other Cas9 are also known (see, e.g., addgene.org/crispr/guide/#pam-table). Guidelines and computer-assisted methods for generating gRNAs are available (see, e.g, CRISPR Design Tool (crispr.mit.edu/); Hsu et al. (2013) Nat. Biotechnol. 31:827-832; addgene.org/CRISPR; and CRISPR gRNA Design tool—DNA2.0 (dna20.com/eCommerce/startCas9)). Typically, the PAM sequence is 3′ of the DNA target sequence in the genomic sequence.

In a particular embodiment, the method comprises administering at least one Cas9 (e.g., the protein and/or a nucleic acid molecule encoding Cas9) and at least one gRNA (e.g., a nucleic acid molecule encoding the gRNA) to the cell or subject. In a particular embodiment, the Cas9 is S. pyogenes Cas9. In a particular embodiment, the targeted PAM is in the 5′UTR, promoter, or first intron. When present, a second gRNA is provided which targets anywhere from the 5′UTR to the 3′UTR of the gene, particularly within the first intron. The nucleic acids of the instant invention may be administered consecutively (before or after) and/or at the same time (concurrently). The nucleic acid molecules may be administered in the same composition or in separate compositions. In a particular embodiment, the nucleic acid molecules are delivered in a single vector (e.g., a viral vector).

In a particular embodiment, the nucleic acid molecules of the instant invention are delivered (e.g., via infection, transfection, electroporation, etc.) and expressed in cells via a vector (e.g., a plasmid), particularly a viral vector. The expression vectors of the instant invention may employ a strong promoter, a constitutive promoter, and/or a regulated promoter. In a particular embodiment, the nucleic acid molecules are expressed transiently. Examples of promoters are well known in the art and include, but are not limited to, RNA polymerase II promoters, the T7 RNA polymerase promoter, and RNA polymerase III promoters (e.g., U6 and H1; see, e.g., Myslinski et al. (2001) Nucl. Acids Res., 29:2502-09). Examples of expression vectors for expressing the molecules of the invention include, without limitation, plasmids and viral vectors (e.g., adeno-associated viruses (AAVs), adenoviruses, retroviruses, and lentiviruses).

In a particular embodiment, the guide RNA of the instant invention may comprise separate nucleic acid molecules. For example, one RNA may specifically hybridize to a target sequence (crRNA) and another RNA (trans-activating crRNA (tracrRNA)) specifically hybridizes with the crRNA. In a particular embodiment, the guide RNA is a single molecule (sgRNA) which comprises a sequence which specifically hybridizes with a target sequence (crRNA; complementary sequence) and a sequence recognized by Cas9 (e.g., a tracrRNA sequence; scaffold sequence).

Examples of gRNA scaffold sequences are well known in the art (e.g., 5′-GUUUUAGAGC UAGAAAUAGC AAGUUAAAAU AAGGCUAGUC CGUUAUCAAC UUGAAAAAGU GGCACCGAGU CGGUGCUUUU; SEQ ID NO: 4). As used herein, the term “specifically hybridizes” does not mean that the nucleic acid molecule needs to be 100% complementary to the target sequence. Rather, the sequence may be at least 80, 85%, 90%, 95%, 97%, 99%, or 100% complementary to the target sequences (e.g., the complementary between the gRNA and the genomic DNA). The greater the complementarity reduces the likelihood of undesired cleavage events at other sites of the genome. In a particular embodiment, the region of complementarity (e.g., between a guide RNA and a target sequence) is at least about 10, at least about 12, at least about 15, at least about 17, at least about 20, at least about 25, at least about 30, at least about 35, or more nucleotides. In a particular embodiment, the region of complementarity (e.g., between a guide RNA and a target sequence) is about 15 to about 25 nucleotides, about 15 to about 23 nucleotides, about 16 to about 23 nucleotides, about 17 to about 21 nucleotides, about 18 to about 22 nucleotides, or about 20 nucleotides. In a particular embodiment, the guide RNA targets a sequence or comprises a sequence (inclusive of RNA version of DNA molecules) as set forth in the Example (e.g., Table 1) provided herein (e.g., sgRNA1, sgRNA2, sgRNA3, sgRNA4, sgRNA5, or sgRNA6; particularly, sgRNA1, sgRNA3, or sgRNA4). In a particular embodiment, the guide RNA targets a sequence or comprises a sequence (e.g., RNA version) which has at least 80%, 85%, 90%, 95%, 97%, 99%, or 100% homology or identity to a sequence set forth in the Example (e.g., sgRNA1, sgRNA2, sgRNA3, sgRNA4, sgRNA5, or sgRNA6; particularly, sgRNA1, sgRNA3, or sgRNA4). The sequences may be extended or shortened by 1, 2, 3, 4, or 5 nucleotides at the end of the sequence opposite from the PAM (e.g., at the 5′ end). When the sequence is extended the added nucleotides should correspond to the genomic sequence.

The above methods also encompass ex vivo methods. For example, the methods of the instant invention can comprise isolating hematopoietic cells (e.g., erythroid precursor cells) or erythroid cells from a subject, delivering at least one PP6C inhibitor to the cells, and administering the treated cells to the subject. The isolated cells (e.g., erythroid cells) may also be treated with other reagents in vitro, such as at least one fetal hemoglobin inducer, prior to administration to the subject.

The methods of the instant invention may further comprise monitoring the disease or disorder in the subject after administration of the composition(s) of the instant invention to monitor the efficacy of the method. For example, the subject may be monitored for characteristics of low hemoglobin or a hemoglobinopathy or thalassemia.

When an inhibitory nucleic acid molecule is delivered to a cell or subject, the inhibitory nucleic acid molecule may be administered directly or an expression vector may be used. In a particular embodiment, the inhibitory nucleic acid molecules are delivered (e.g., via infection, transfection, electroporation, etc.) and expressed in cells via a vector (e.g., a plasmid), particularly a viral vector. The expression vectors of the instant invention may employ a strong promoter, a constitutive promoter, and/or a regulated promoter. In a particular embodiment, the inhibitory nucleic acid molecules are expressed transiently. In a particular embodiment, the promoter is cell-type specific (e.g., erythroid cells). Examples of promoters are well known in the art and include, but are not limited to, RNA polymerase II promoters, the T7 RNA polymerase promoter, and RNA polymerase III promoters (e.g., U6 and H1; see, e.g., Myslinski et al. (2001) Nucl. Acids Res., 29:2502-09). Examples of expression vectors for expressing the molecules of the invention include, without limitation, plasmids and viral vectors (e.g., adeno-associated viruses (AAVs), adenoviruses, retroviruses, and lentiviruses).

As explained hereinabove, the compositions of the instant invention are useful for increasing hemoglobin production and for treating hemoglobinopathies and thalassemias. A therapeutically effective amount of the composition may be administered to a subject in need thereof. The dosages, methods, and times of administration are readily determinable by persons skilled in the art, given the teachings provided herein.

The components as described herein will generally be administered to a patient as a pharmaceutical preparation. The term “patient” or “subject” as used herein refers to human or animal subjects. The components of the instant invention may be employed therapeutically, under the guidance of a physician for the treatment of the indicated disease or disorder.

The pharmaceutical preparation comprising the components of the invention may be conveniently formulated for administration with an acceptable medium (e.g., pharmaceutically acceptable carrier) such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the agents to be administered, its use in the pharmaceutical preparation is contemplated.

The compositions of the present invention can be administered by any suitable route, for example, by injection (e.g., for local (direct) or systemic administration), oral, pulmonary, topical, nasal or other modes of administration. The composition may be administered by any suitable means, including parenteral, intramuscular, intravenous, intraarterial, intraperitoneal, subcutaneous, topical, inhalatory, transdermal, intrapulmonary, intraareterial, intrarectal, intramuscular, and intranasal administration. In a particular embodiment, the composition is administered directly to the blood stream (e.g., intravenously). In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington: The Science and Practice of Pharmacy, 21st edition, Philadelphia, Pa. Lippincott Williams & Wilkins. The pharmaceutical composition of the present invention can be prepared, for example, in liquid form, or can be in dried powder form (e.g., lyophilized for later reconstitution).

As used herein, “pharmaceutically acceptable carrier” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the molecules to be administered, its use in the pharmaceutical preparation is contemplated.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous. Injectable suspensions may be prepared, in which case appropriate liquid carriers, suspending agents and the like may be employed. Pharmaceutical preparations for injection are known in the art. If injection is selected as a method for administering the therapy, steps should be taken to ensure that sufficient amounts of the molecules reach their target cells to exert a biological effect.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient.

Appropriate concentrations for alleviation of a particular pathological condition may be determined by dosage concentration curve calculations, as known in the art. The appropriate dosage unit for the administration of the molecules of the instant invention may be determined by evaluating the toxicity of the molecules in animal models. Various concentrations of pharmaceutical preparations may be administered to mice with transplanted human tumors, and the minimal and maximal dosages may be determined based on the results of significant reduction of tumor size and side effects as a result of the treatment. Appropriate dosage unit may also be determined by assessing the efficacy of the treatment in combination with other standard therapies.

The pharmaceutical preparation comprising the molecules of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

### Definitions

The singular forms “a,” “an,” and “the” include plural referents unless the context clearly dictates otherwise.

The terms “isolated” is not meant to exclude artificial or synthetic mixtures with other compounds or materials, or the presence of impurities that do not interfere with the fundamental activity, and that may be present, for example, due to incomplete purification, or the addition of stabilizers.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in Remington: The Science and Practice of Pharmacy, (Lippincott, Williams and Wilkins); Liberman, et al., Eds., Pharmaceutical Dosage Forms, Marcel Decker, New York, N.Y.; and Rowe, et al., Eds., Handbook of Pharmaceutical Excipients, Pharmaceutical Pr.

The term “treat” as used herein refers to any type of treatment that imparts a benefit to a patient suffering from an injury, including improvement in the condition of the patient (e.g., in one or more symptoms), delay in the progression of the condition, etc.

As used herein, the term “prevent” refers to the prophylactic treatment of a subject who is at risk of developing a condition and/or sustaining an injury, resulting in a decrease in the probability that the subject will develop conditions associated with the hemoglobinopathy or thalassemia.

A “therapeutically effective amount” of a compound or a pharmaceutical composition refers to an amount effective to prevent, inhibit, or treat a particular injury and/or the symptoms thereof. For example, “therapeutically effective amount” may refer to an amount sufficient to modulate the pathology associated with a hemoglobinopathy or thalassemia.

As used herein, the term “subject” refers to an animal, particularly a mammal, particularly a human.

A “vector” is a genetic element, such as a plasmid, cosmid, bacmid, phage, transposon, or virus, to which another genetic sequence or element (either DNA or RNA) may be attached so as to bring about the replication and/or expression of the attached sequence or element. A vector may be either RNA or DNA and may be single or double stranded. A vector may comprise expression operons or elements such as, without limitation, transcriptional and translational control sequences, such as promoters, enhancers, translational start signals, polyadenylation signals, terminators, and the like, and which facilitate the expression of a polynucleotide or a polypeptide coding sequence in a host cell or organism.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, amino acids, or nucleic acids.

An “antibody” or “antibody molecule” is any immunoglobulin, including antibodies and fragments thereof, that binds to a specific antigen. As used herein, antibody or antibody molecule contemplates intact immunoglobulin molecules, immunologically active portions/fragment (e.g., antigen binding portion/fragment) of an immunoglobulin molecule, and fusions of immunologically active portions of an immunoglobulin molecule. Antibody fragments include, without limitation, immunoglobulin fragments including, without limitation: single domain (Dab; e.g., single variable light or heavy chain domain), Fab, Fab′, F(ab′)₂, and F(v); and fusions (e.g., via a linker) of these immunoglobulin fragments including, without limitation: scFv, scFv₂, scFv-Fc, minibody, diabody, triabody, and tetrabody.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

The phrase “small, interfering RNA (siRNA)” refers to a short (typically less than 30 nucleotides long, particularly 12-30 or 20-25 nucleotides in length) double stranded RNA molecule. Typically, the siRNA modulates the expression of a gene to which the siRNA is targeted. Methods of identifying and synthesizing siRNA molecules are known in the art (see, e.g., Ausubel et al., Current Protocols in Molecular Biology, John Wiley and Sons, Inc). Short hairpin RNA molecules (shRNA) typically consist of short complementary sequences (e.g., an siRNA) separated by a small loop sequence (e.g., 6-15 nucleotides, particularly 7-10 nucleotides) wherein one of the sequences is complimentary to the gene target. shRNA molecules are typically processed into an siRNA within the cell by endonucleases. Exemplary modifications to siRNA molecules are provided in U.S. Application Publication No. 20050032733. For example, siRNA and shRNA molecules may be modified with nuclease resistant modifications (e.g., phosphorothioates, locked nucleic acids (LNA), 2′-O-methyl modifications, or morpholino linkages). Expression vectors for the expression of siRNA or shRNA molecules may employ a strong promoter which may be constitutive or regulated. Such promoters are well known in the art and include, but are not limited to, RNA polymerase II promoters, the T7 RNA polymerase promoter, and the RNA polymerase III promoters U6 and H1 (see, e.g., Myslinski et al. (2001) Nucl. Acids Res., 29:2502-09).

“Antisense nucleic acid molecules” or “antisense oligonucleotides” include nucleic acid molecules (e.g., single stranded molecules) which are targeted (complementary) to a chosen sequence (e.g., to translation initiation sites and/or splice sites) to inhibit the expression of a protein of interest. Such antisense molecules are typically between about 15 and about 50 nucleotides in length, more particularly between about 15 and about 30 nucleotides, and often span the translational start site of mRNA molecules. Antisense constructs may also be generated which contain the entire sequence of the target nucleic acid molecule in reverse orientation. Antisense oligonucleotides targeted to any known nucleotide sequence can be prepared by oligonucleotide synthesis according to standard methods. Antisense oligonucleotides may be modified as described above to comprise nuclease resistant modifications.

The following example is provided to illustrate various embodiments of the present invention. It is not intended to limit the invention in any way.

### Example

Hemoglobin is comprised of a tetramer containing two α-type and two β-type subunits. The β-type globin gene cluster consists of an embryonic (ε-globin), two fetal (γ-globin) and two adult type (δ-globin and β-globin) genes. Mutations in the β-globin gene that underlie sickle cell disease (SCD) and some types of β-thalassemia become clinically relevant after birth when the fetal genes are silenced. Increased fetal hemoglobin (HbF) production due to natural genetic variation or therapeutic intervention can lower morbidity and mortality in β-hemoglobinopathies (Platt, et al., N. Engl. J. Med. (1994) 330:1639-1644; Miller, et al., N. Engl. J. Med. (2000) 342:83-89). While promising strategies involving gene addition or genome editing are being pursued (Traxler, et al., Nat. Med. (2016) 22:987-990; Canver, et al., Blood (2016) 127:2536-2545; Cai, et al., Stem Cells Transl. Med. (2018) 7:87-97; Wienert, wt al., Trends Genet. (2018) 34(12):P927-940), their implementation will be largely restricted to patients with access to sophisticated medical providers. Effective HbF induction by pharmacologic means is therefore needed, but remains a challenge.

BCL11A and LRF (ZBTB7A) are critical direct transcriptional repressors of the γ-globin genes (Menzel, et al., Nat. Genet. (2007) 39:1197-1199; Uda, et al., Proc. Natl. Acad. Sci. (2008) 105:1620-1625; Sankaran, et al., Science (2008) 322:1839-1842; Masuda, et al., Science (2016) 351:285-289). However, as DNA binding proteins with functions in multiple tissues, they remain difficult to target pharmacologically and in an erythroid selective manner. Moreover, LRF depletion delays erythroid differentiation (Masuda, et al., Science (2016) 351:285-289; Maeda, et al., Dev. Cell. (2009) 17:527-540). In spite of a deep understanding of the transcriptional control of the globin genes, the regulatory circuitry outside of DNA binding nuclear factors remains under-explored. Yet, it is clear from targeted depletion experiments that non-DNA binding co-regulatory complexes can play pivotal roles in HbF silencing (Liu, et al., Cell (2017) 170:1028-1043; Grevet, et al., Science (2018) 361:285-290).

A CRISPR screening strategy (Shi et al. (2015) Nat. Biotechnol., 33(6):661-7; Shalem et al., Nat. Rev. Genet. (2015) 16(5):299-311) was employed to identify regulators of fetal globin expression. Clustered, regularly interspaced, short palindromic repeat (CRISPR)/Cas9 technology is well known in the art (see, e.g., Sander et al. (2014) Nature Biotech., 32:347-355; Jinek et al. (2012) Science, 337:816-821; Cong et al. (2013) Science 339:819-823; Ran et al. (2013) Nature Protocols 8:2281-2308; Mali et al. (2013) Science 339:823-826). Cas9 possesses two nuclease domains, a RuvC-like nuclease domain and a HNH-like nuclease domain, and is responsible for the destruction of the target DNA (Jinek et al. (2012) Science, 337:816-821; Sapranauskas et al. (2011) Nucleic Acids Res. 39:9275-9282). The two nucleases generate double-stranded breaks. The double-stranded endonuclease activity of Cas9 requires a target sequence (e.g., ˜20 nucleotides, see above) and a short conserved sequence (˜2-5 nucleotides; e.g., 3 nucleotides) known as protospacer-associated motif (PAM), which follows immediately 3′-of the CRISPR RNA (crRNA) complementary sequence (Jinek et al. (2012) Science, 337:816-821; Nishimasu et al. (2014) Cell 156(5):935-49; Swarts et al. (2012) PLoS One, 7:e35888; Sternberg et al. (2014) Nature 507(7490):62-7). The double strand break can be repaired by non-homologous end joining (NHEJ) pathway yielding an insertion and/or deletion or, in the presence of a donor template, by homology-directed repair (HDR) pathway for replacement mutations (Overballe-Petersen et al. (2013) Proc. Natl. Acad. Sci. U.S.A. 110:19860-19865; Gong et al. (2005) Nat. Struct. Mol. Biol. 12:304-312). The RNA-guided CRISPR/Cas9 system involves expressing Cas9 along with a guide RNA molecule (gRNA). When coexpressed, gRNAs bind and recruit Cas9 to a specific genomic target sequence where it mediates a double strand DNA (dsDNA) break and activates the dsDNA break repair machinery. Specific DNA fragments can be deleted when two gRNA/Cas9 complexes generate dsDNA breaks at relative proximity, and the genomic DNA is repaired by nonhomologous end joining.

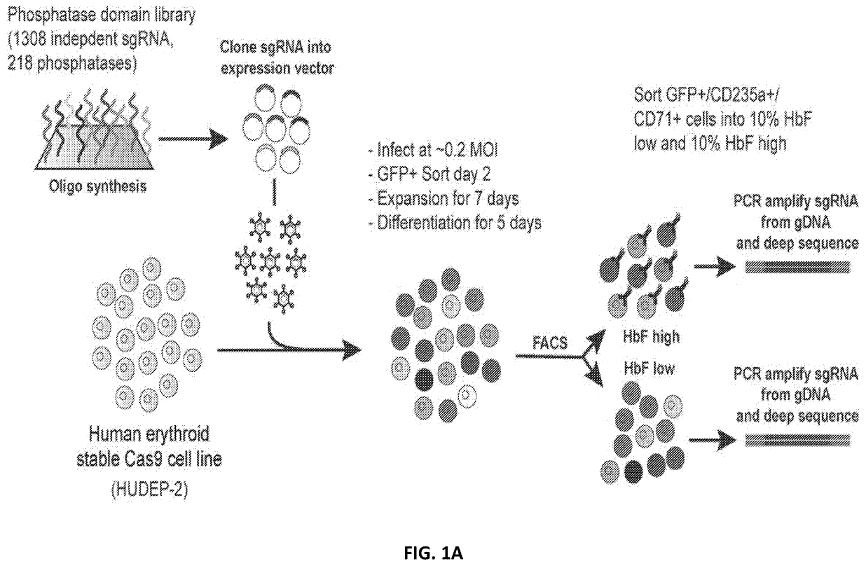

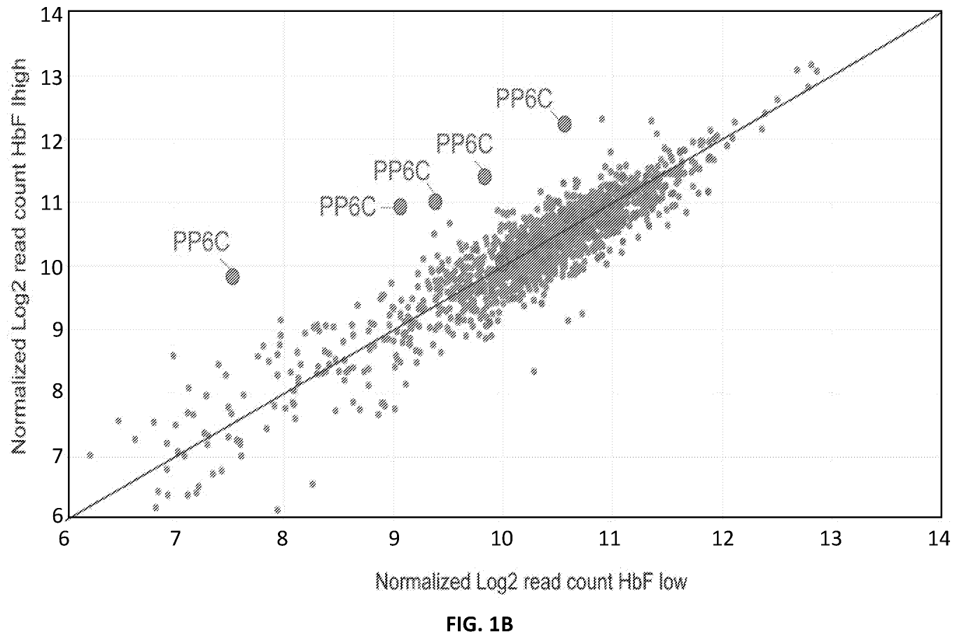

sgRNAs targeting functional protein domains generates phenotype-altering mutations at a higher rate compared to sgRNAs designed to generate null alleles (Shi, et al., Nat. Biotechnol. (2015) 33:661-667). In order to identify additional potential γ-globin repressors, a library of sgRNAs targeting 218 different phosphatase domains with 6 guides per domain (i.e. 1308 independent guides). This library also included non-targeting sgRNAs as negative controls. This sgRNA library was cloned into the LRG 2.1T lentiviral vector (Grevet et al., Science (2018) 361(6399):285-290) and then introduced into human umbilical cord blood-derived erythroid progenitor (HUDEP)-2 cells (Kurita, et al., PLoS One (2013) 8:e59890) stably expressing Cas9 (FIG. 1A). Cells were stained using an anti-HbF antibody and fluorescence-activated cell sorting (FACS) was used to isolate the top 10% and bottom 10% of HbF expressing cells and the sgRNAs from each population was deep sequenced (FIG. 1A; Canver et al. (2015) Nature 527(7577):192-197). FIG. 1B shows that 5 of 6 sgRNAs targeting PP6C resulted in enriched fetal globin expression as compared to non-targeting sgRNAs, indicating that it functions as a repressor of γ-globin.

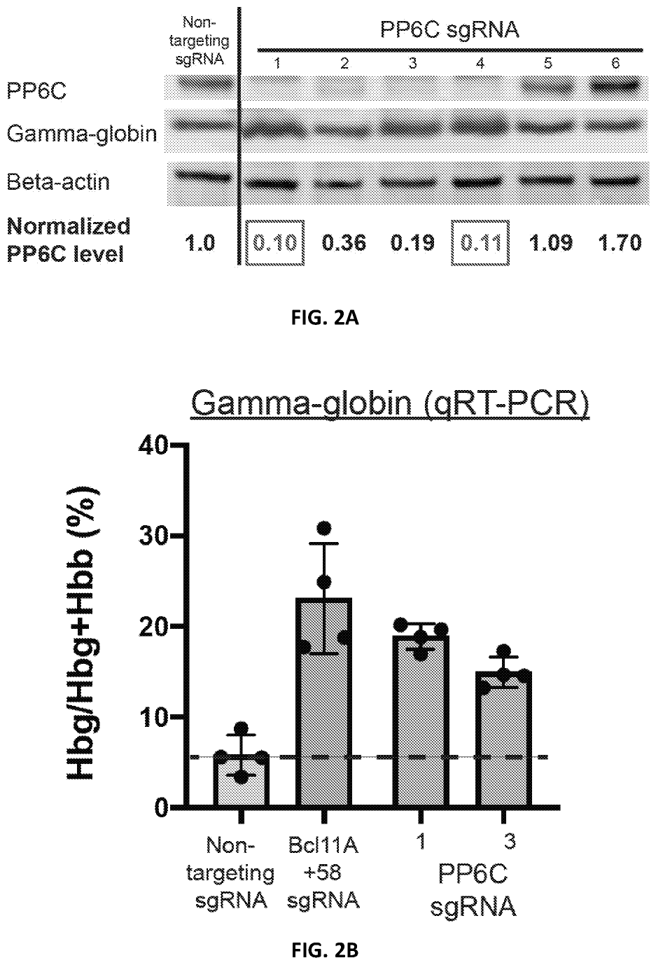

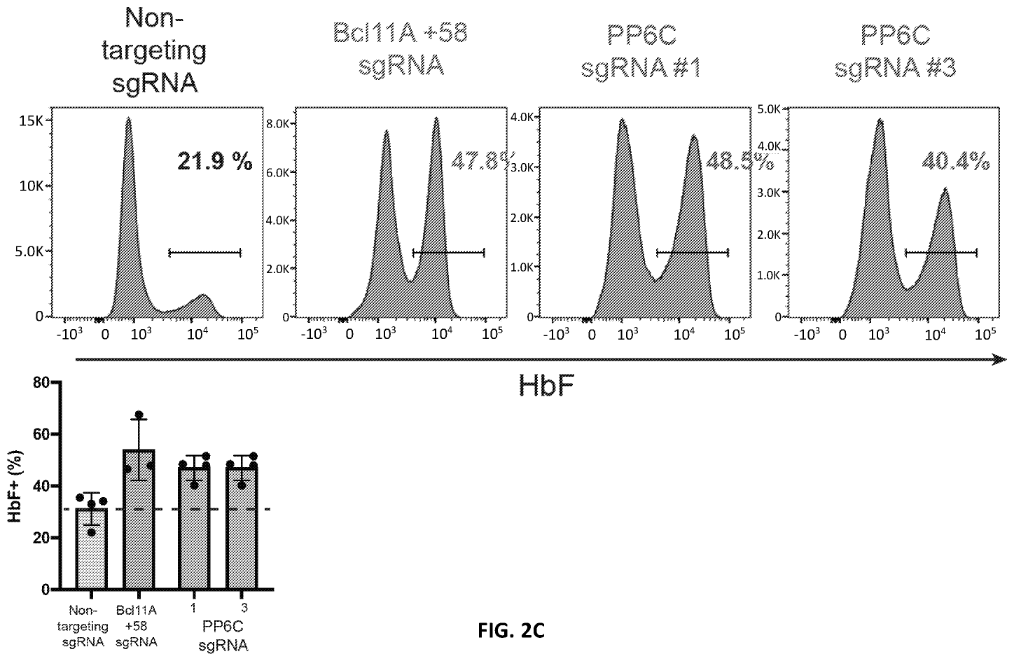

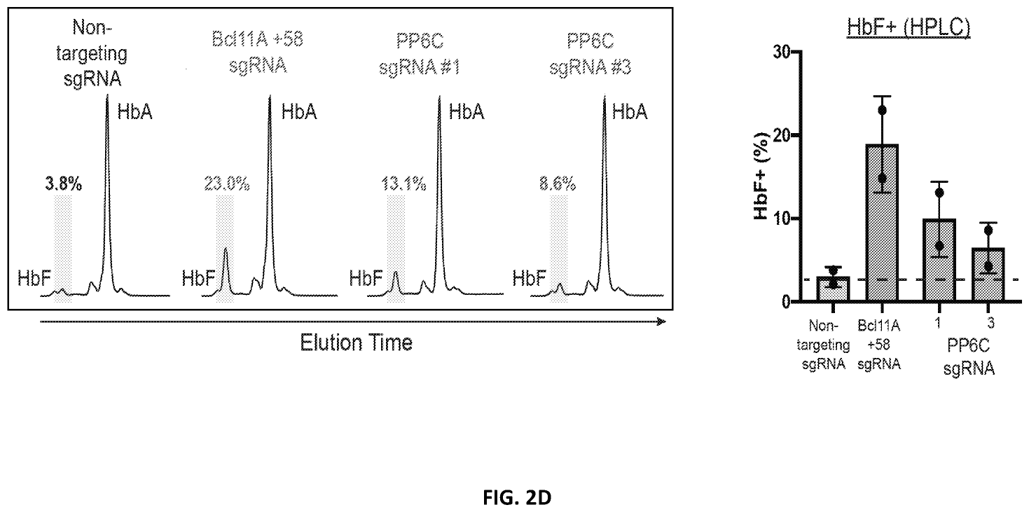

These results were confirmed in primary human cells by utilizing RNP-based electroporation of SpCas9 combined with guide RNA targeting PP6C. Cells were transduced with one of the six guide RNAs targeting PP6C, a guide RNA targeting BCL11A exon 2 (positive control; BCL11A is a repressor of HbF expression), or non-targeting guide RNA or untreated control (negative controls). FIG. 2A shows that PP6C protein level is significantly depleted utilizing PP6C guide RNA #1, #3, and #4, and that these samples show significant induction in gamma-globin.

These specific sgRNAs were utilized for further primary human cell testing. A quantitative reverse transcription PCR (RT-qPCR) analysis was subsequently performed on the above cells to measure mRNA levels. FIG. 2B shows that PP6C sgRNAs significantly increased gamma-globin RNA levels compared to the non-targeting and untreated controls and achieved gamma-globin levels comparable to targeting of the BCL11A +58 enhancer. As seen in FIG. 2C, flow cytometric analysis with an HbF antibody shows that the PP6C sgRNAs also significantly increased expression of HbF compared to the negative controls. Finally, cation-exchange HPLC, which serves as the clinical standard for quantifying HbF protein levels, was performed. FIG. 2D shows that PP6C sgRNAs achieve 2-3-fold induction of HbF by HPLC. Taken together, these data confirm in primary human erythroid cells that depletion of PP6C leads to a robust increase in HbF.

TABLE 1
sgRNA sequences

| CRISPR sgRNA name | Sequence |
|---|---|
| PP6C sgRNA1 | GCACATCAACTAGTGCACGA |
| | (SEQ ID NO: 5) |
| PP6C sgRNA2 | CCTAATTACTGCTATCGTTG |
| | (SEQ ID NO: 6) |
| PP6C sgRNA3 | GGAACTGCCCGGAATAACTT |
| | (SEQ ID NO: 7) |
| PP6C sgRNA4 | TCCTCGGGGACTGATAGCCC |
| | (SEQ ID NO: 8) |
| PP6C sgRNA5 | AATTTCCTGATTCCGTTCGA |
| | (SEQ ID NO: 9) |
| PP6C sgRNA6 | CGCAAAAGTGTAATACGATC |
| | (SEQ ID NO: 10) |
| Neg sgRNA | GCACTACCAGAGCTAACTCA |
| | (SEQ ID NO: 11) |
| Bcll1A sgRNA | CTAACAGTTGCTTTTATCAC |
| | (SEQ ID NO: 12) |

When measuring the transcriptional effects of PP6C perturbation, robust increases in γ-globin mRNA and pre-mRNA levels were observed, indicating that PP6C impinges on transcriptional regulation of γ-globin. In summary, PP6C functions as a novel regulator of HbF and offers a therapeutic target for monotherapy or combination therapies for γ-hemoglobinopathies.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1: A method of increasing the level of human fetal hemoglobin in a cell or subject, the method comprising administering at least one protein phosphatase 6 catalytic subunit (PP6C) inhibitor to the cell or subject.

2: The method of claim 1, wherein the subject has a β-chain hemoglobinopathy.

3: The method of claim 1, wherein the subject has thalassemia.

4: The method of claim 1, wherein the subject has sickle cell disease.

5: The method of claim 1, wherein the PP6C inhibitor is a small molecule.

6: The method of claim 1, wherein the PP6C inhibitor is a phosphatase inhibitor or a heme binding domain inhibitor.

7: The method of claim 1, wherein the PP6C inhibitor is an inhibitory nucleic acid molecule.

8: The method of claim 1, wherein the cell is an erythroid cell.

9: The method of claim 1, further comprising administering at least one fetal hemoglobin inducer to the cell or subject.

10: The method of claim 9, wherein said fetal hemoglobin inducer is pomalidomide.

11: A method of treating a hemoglobinopathy in a subject in need thereof, the method comprising administering a composition comprising at least one protein phosphatase 6 catalytic subunit (PP6C) inhibitor and a pharmaceutically acceptable carrier to the subject.

12: The method of claim 11, wherein the subject has a β-chain hemoglobinopathy.

13: The method of claim 11, wherein the subject has thalassemia.

14: The method of claim 11, wherein the subject has sickle cell anemia.

15: The method of claim 11, wherein the PP6C inhibitor is a small molecule.

16: The method of claim 11, wherein the PP6C inhibitor is a phosphatase inhibitor or a heme binding domain inhibitor.

17: The method of claim 11, wherein the PP6C inhibitor is an inhibitory nucleic acid molecule.

18: The method of claim 11, further comprising administering at least one fetal hemoglobin inducer to the subject.

19: The method of claim 18, wherein said fetal hemoglobin inducer is pomalidomide.

20: The method of claim 11, wherein the PP6C inhibitor is contained within a cell administered to the subject.

21: A composition comprising at least one protein phosphatase 6 catalytic subunit (PP6C) inhibitor and at least one fetal hemoglobin inducer.

22: The composition of claim 21, wherein said fetal hemoglobin inducer is pomalidomide.


SEQUENCE LISTING (12 sequences)

SEQ ID NO: 1 - protein, 305 aa, Homo sapiens
MAPLDLDKYVEIARLCKYLPENDLKRLCDYVCDLLLEESNVQPVSTPVTVCGDIHGQFYDLCELFRTGGQVPDTNYIFMGDFVDRGYYSLETFTYLLALKAKWPDRITLLRGNHESRQITQVYGFYDECQTKYGNANAWRYCTKVFDMLTVAALIDEQILCVHGGLSPDIKTLDQIRTIERNQEIPHKGAFCDLVWSDPEDVDTWAISPRGAGWLFGAKVTNEFVHINNLKLICRAHQLVHEGYKFMFDEKLVTVWSAPNYCYRCGNIASIMVFKDVNTREPKLFRAVPDSERVIPPRTTTPYFL

SEQ ID NO: 2 - DNA, 918 bp, Homo sapiens
atggcgccgctagacctggacaagtatgtggaaatagcgcggctgtgcaagtacctgccagagaacgacctgaagcggctatgtgactacgtttgtgacctcctcttagaagagtcaaatgttcagccagtatcaacaccagtaacagtgtgtggagatatccatggacagttttatgacctttgtgaactgttcagaactggaggtcaggttcctgacacaaactacatatttatgggtgattttgtagacagaggttactatagtttggagaccttcacttaccttcttgcattaaaggctaaatggcctgatcgtattacacttttgcgaggaaatcatgagagtagacagataacacaggtctatggattttatgatgagtgccaaaccaaatatggaaatgctaatgcctggagatactgtaccaaagtttttgacatgctcacagtagcagctttaatagatgagcagattttgtgtgtccatggtggtttatctcctgatatcaaaacactggatcaaattcgaaccatcgaacggaatcaggaaattcctcataaaggagcattttgtgatctggtttggtcagatcctgaagatgtggatacctgggctatcagtccccgaggagcaggttggctttttggagcaaaggtcacaaatgagtttgttcatatcaacaacttaaaactcatctgcagagcacatcaactagtgcacgaaggctataaatttatgtttgatgagaagctggtgacagtatggtctgctcctaattactgctatcgttgtggaaatattgcttcgatcatggtcttcaaagatgtaaatacaagagaaccaaagttattccgggcagttccagattcagaacgtgttattcctcccagaacgacaacgccatatttcctttga

SEQ ID NO: 3 - DNA, 4367 bp, Homo sapiens
gaagcgggctctcttggggcctgcagtcggggttcagccagagggggcgccgggcgtaccgacgtagcggggagaaacgggtcgcgctttaacggcgctgcgcaggcgccactcacgggccggacgtgacgcagggaaagttccggcttcggcctccgccgctgccgccgccgctgctacagccgccgccgccgctgttgccgcggcttgttattcttaaaatggcgccgctagacctggacaagtatgtggaaatagcgcggctgtgcaagtacctgccagagaacgacctgaaggtgagccctatttgcgggctcgccccttccggctgtggggcgcccgccggccggcccttcctttctccaggacccccgccggtatttcattttcttcgttttctaaaggagcggctatgtgactacgtttgtgacctcctcttagaagagtcaaatgttcagccagtatcaacaccagtaacagtgtgtggagatatccatggacagttttatgacctttgtgaactgttcagaactggaggtcaggttcctgacacaaactacatatttatgggtgattttgtagacagaggttactatagtttggagaccttcacttaccttcttgcattaaaggctaaatggcctgatcgtattacacttttgcgaggaaatcatgagagtagacagataacacaggtctatggattttatgatgagtgccaaaccaaatatggaaatgctaatgcctggagatactgtaccaaagtttttgacatgctcacagtagcagctttaatagatgagcagattttgtgtgtccatggtggtttatctcctgatatcaaaacactggatcaaattcgaaccatcgaacggaatcaggaaattcctcataaaggagcattttgtgatctggtttggtcagatcctgaagatgtggatacctgggctatcagtccccgaggagcaggttggctttttggagcaaaggtcacaaatgagtttgttcatatcaacaacttaaaactcatctgcagagcacatcaactagtgcacgaaggctataaatttatgtttgatgagaagctggtgacagtatggtctgctcctaattactgctatcgttgtggaaatattgcttcgatcatggtcttcaaagatgtaaatacaagagaaccaaagttattccgggcagttccagattcagaacgtgttattcctcccagaacgacaacgccatatttcctttgaggccttcgcccatcctgctgacccatttttctgccctcttcttaccccaattttcttgtattaccctctacaatatactttttattgagcactttgctgctgaaatgctgcctcttgcctttttttttttaaattttaaattatctaaatttattgtttgttgtggtgtctatagcaaagtttttctatcaattttcccccatcccatccccaccctggactcatttgagaagacttgagaaatgtcttaatactcacactgctgcatgtagctcttgcttatttactggtctgggaaacaggatgtgtttcctttttttaaaagccaattgacagattacacctaaatactcctccttttgtatcattcagccttttgttttagtttggtaagttttaagaaatttcagcagcaaagttgttattcagtgggcacgatggactccaaatgcctcaagttatgtatacctgtcccagatgtaaacttcattgtcctttgttggatgatattttaaatggatataaaataaattggtctaaagggctgccctccttgttgtgtttttaaattttagttaaaaactgctacagcttatgactttgtactttaagataattgtattgatcttttttcagattccttgtattttttaataaagtaatcttaaataaaactcagataggttaagtgttagaaattttaaacagcttacattgttagcgtaaagttatcttttcttttttcctaatcagagttcttgaccctttggttattgagtttaaaacttcaattgaaattcaatagtatttatttttgaaaaaaatcactaaactgtgcctaaagaacataactgccatattaatgttttggtttatatcctctatagtaatagaaaaacatttaatacttgtaatgctgatgtgttaatttgataccagttgagtagaatgtgatcaatccagtttacaatctatcatgagtattattaactaaaatctatgtgcttttcaataggaatcattcttctcttgctgtaacacttgaccttaacttttagaaagtgttcatttttaaactgcaactggaaaggttgaaaagttaggactcttgtatttgtgaactgtaatctgaagcagattatttaaagtgtagaaaaagaaacaagttcttcttttttgcaaaggtctgtgataccatatttcagctttgtgtaagtaatttgaatatccaaagggttgtgatgatcagttctgaatatgcaactgtccacttaataaggacaagtattccagtatctcttatgactgtagtcataaatgatgttggaatgtacattttgtgaaatagttggtatccctttactatgattaatttttgttattccaggaaatacttgtgaagccagccaattaataaagcactttagcatctgttcaggtagttttgaaaacccacttttccccttcaggataagaacttccaggttacctaaaaatgcaataaaaatctttatagtctaagcttcttggcatataatttttcatcagggttttcttttattaattgagcacaatactgttttgatttaatttttttccctcaaaccacccagctccttacatagtttttaattatatagctatatgaaagaggtggctaagacatttgctgcacacacttgaactaatgttgggtcagtagcttcgtgttactgccctgatcccagtgtaattcaggtggaaaggtatttttatctcacagggatatgtagctatgtattttactaattgtgaaacactggaaattaatgatgcagacaacttggtgtggtctttgaacagctctctgcagtatttttttttttcctgttaccataaattgtatttaagtgcttgctttccacttaagttgtactaatagaaccggtaactcccagccctccctgtttgacactccttagcttagattgatgtagttgtttttgttatccctataatgtgacttttatttttaagtcattgtgtactgcatttgtttgtcactagttgggcacgtgccaataatattctctccattccttatctgccatctctgttttgcctgatttctcttcaactgaataatggctttttgcatggaaaaaatagtttttactattagacgtgtaaagggaagagagagctaatgtattggactttgtgagcctacaaggaatattttggatccctccaataaataagggctatgtactatatgtactatatagagttatcatgtggtggaagatacttgcaagtcatagatttatgggcaggaggatttgttactccctatatctaggctgaatgtaaaatcccttatgttgtatcaatgggggtaaaaactatttttatttgcctatgatatacttggtttctaataaagtgccctaggctctagtgagaactgtctactttgaattgccatttactccctttcctcctttggccgatatgctcttggctagctttttataagttaatgtgtttccccaaaaagtttcactactttatattcatttgagtgtgatcctaaaacacctggatcaacagtacatctcatatgcaatctgcatcagctcctattctgtctggatgtctagaactgttggaagattttgacgtcttaagccctaggttttgctttgggaaataaggtttgaaatattgttcattgcattaagatttgtgtgtgtgtgctttgtaagcccagaaccaggttttggaaaatgcctgtactgtgaaagcaaattaggactctttctgagcctctttcattgtcagaaatagaatcactttccatcagcttccaggaaattgtgtatctggagtcgaagagatttgacatcaagaatccagatttttaaatgtaattgttttttaaatgctaatgtttgtaaagcaccttcagttcttcggatgaaaggtgctatattccctcagtgtaaattaataaaaagattacaggaagtttgtcaaaaaattcaatgcatagtctgtagtatgtcctgacaagaagttagcattttatataagaaattaaaaaatgcttattcctccaaaaaaaaaaaaaaaaaa

SEQ ID NO: 4 - RNA, 80 nt, Artificial Sequence
  guide RNA scaffold sequence
guuuuagagcuagaaauagcaaguuaaaauaaggcuaguccguuaucaacuugaaaaaguggcaccgagucggugcuuuu

SEQ ID NO: 5 - DNA, 20 bp, Artificial Sequence
  sgRNA1
gcacatcaactagtgcacga

SEQ ID NO: 6 - DNA, 20 bp, Artificial Sequence
  sgRNA2
cctaattactgctatcgttg

SEQ ID NO: 7 - DNA, 20 bp, Artificial Sequence
  sgRNA3
ggaactgcccggaataactt

SEQ ID NO: 8 - DNA, 20 bp, Artificial Sequence
  sgRNA4
tcctcggggactgatagccc

SEQ ID NO: 9 - DNA, 20 bp, Artificial Sequence
  sgRNA5
aatttcctgattccgttcga

SEQ ID NO: 10 - DNA, 20 bp, Artificial Sequence
  sgRNA6
cgcaaaagtgtaatacgatc

SEQ ID NO: 11 - DNA, 20 bp, Artificial Sequence
  neg sgRNA
gcactaccagagctaactca

SEQ ID NO: 12 - DNA, 20 bp, Artificial Sequence
  BCL11A sgRNA
ctaacagttgcttttatcac